(19) United States
(12) Patent Application Publication
(10) Pub. No.: US 2020/0405992 A1
(43) Pub. Date: Dec. 31, 2020

(54) CONNECTOR WITH A MOUNT FOR A SENSOR

(71) Applicant: Drägerwerk AG & Co. KGaA, Lübeck (DE)

(72) Inventors: Ludger TAPPEHORN, Lübeck (DE); Jan-Henning LÜTKHOFF, Lübeck (DE)

(21) Appl. No.: 16/913,602

(22) Filed: Jun. 26, 2020

(30) Foreign Application Priority Data
Jun. 28, 2019 (DE) 102019004508.4

(51) Int. Cl. A61M 16/08

(52) U.S. Cl. CPC A61M 16/0816; A61M 2016/0027

(57) ABSTRACT

A connector (100) for arranging at least one sensor at a ventilation system. The connector includes at least one basic body (103) for connection to at least one ventilation device, at least one pivoting element (101) for connection to a user, and at least one sensor mount (125) for at least one sensor. The at least one pivoting element (101) is connected directly and undetachably to the basic body (103) on a first side (115). The at least one pivoting element (101) is pivotable about at least one axis with respect to at least one part of the basic body (103) in order to protect the basic body (103) against pulling and compressive forces imparted by the user.

### CROSS REFERENCE TO RELATED APPLICATIONS

This application claims the benefit of priority under 35 U.S.C. § 119 of German Application 10 2019 004 508.4, filed Jun. 28, 2019, the entire contents of which are incorporated herein by reference.

### TECHNICAL FIELD

The present invention pertains to a connector for connecting at least one ventilation device to a user and to a connector for use during the ventilation of a user.

### TECHNICAL BACKGROUND

For the optimal treatment or supply of users requiring ventilation, for example, patients, ventilators or ventilation devices are equipped, as a rule, with various possibilities for detecting user-dependent parameters. These include, for example, a volume flow measurement for determining a respiratory minute volume, a pressure measurement for determining an airway pressure as well as a measuring function for determining the carbon dioxide concentration in the breathing gas.

An operator, for example, a physician, is provided with important information on the status or the metabolism of a user, for example, a patient, especially by the determination of the carbon dioxide concentration.

It is known that a sensor can be inserted as an additional component into a connection line between a ventilation device and a user for measuring the respiratory minute volume and the carbon dioxide concentration, as a result of which the dead space of a resulting global system increases.

The part of a ventilation system through which breathing gas flows during an exhalation and during an inhalation is called dead space in this connection.

Connectors, which guide an air stream (gas stream) from a ventilation device to the user, are used, as a rule, during the ventilation of a user. Such connectors may be configured, for example, as angle connectors in order to make possible an ergonomic arrangement at the user.

Situations in which the connector is subject to a pulling and/or compressive force due to movements of the user will occur, as a rule, during the use of a connector for mechanical ventilation.

A connector, which guarantees rotatability of a tube system, is known from US 2015/040898 A1. The connector is configured in this case as a modular unit, which is provided on a user side and on a device side with an interface each, at which additional devices can be arranged.

A cuvette with two measuring windows located opposite each other for detecting a carbon dioxide concentration in an air stream flowing through a ventilation system is described in US 2009/320846 A1 of Dragerwerk AG & Co. KGaA.

A modular arrangement of sensors or a series arrangement of sensors, i.e., an arrangement of sensors and connectors in a row, leads, as a rule, to an increase in the dead space in a ventilation system and/or to the measured values being influenced by additional components, for example, filters, in an air stream to be measured.

### SUMMARY

Based on this state of the art, a basic object of the present invention is to provide a connector for connecting at least one ventilation device to a user, which overcomes these drawbacks at least partially. Therefore, an object of the present invention is to provide a connector, which makes possible a reliable and accurate checking of a quality of an air stream (gas stream) for ventilating a user.

A connector for arranging at least one sensor at a ventilation system is thus presented. The connector comprises at least one basic body for connection to at least one ventilation device, at least one pivoting element for connection to a user, and at least one mount for at least one sensor. The at least one pivoting element is connected on a first side directly and undetachably to the basic body and is pivotable about at least one axis with respect to at least one part of the basic body in order to protect the basic body from pulling or compressive forces imparted by the user. The connector defines a flow passage, particularly a flow passage for an air stream (a gas stream) flowing through the basic body and the pivoting element.

A direct connection is defined in the context of the present disclosure as a connection without an additional element arranged in between. A sequence of an arrangement comprising the first element and the second element, in which arrangement the first elements follows the second element directly, is absolutely predefined by a direct connection between a first element and a second element.

A mount is defined in the context of the present invention as a connection element, which makes it possible for a sensor to measure an air stream flowing through the connector according to the present invention at a predefined fixed point at the connector. A mount may connect a sensor directly to the connector by the sensor being inserted into the mount, or it may supply the sensor with a pressure column, for example, via at least one connection element.

The connector being presented is used especially to determine the quality of an air stream provided for ventilating a user with a minimal dead space. Provisions are made for this purpose for the at least one pivoting element provided according to the present invention to be connected on a first side to the at least one basic body provided according to the present invention directly and undetachably but pivotably, so that the pivoting element is arranged in a predefined position relative to the basic body and thus there is, in particular, only one degree of freedom with respect to the relative position of the pivoting element relative to the basic body due to a pivotability but not due to a change in position. In particular, the pivoting element may be configured as a ball-and-socket joint pivotable in a plurality of directions or with a plurality of degrees of freedom. The direct connection of the pivoting element to the basic body correspondingly prevents the arrangement of additional components, for example, filters, between the pivoting element and the basic body, so that the volume of the connector is constant independently from a position in a particular ventilation system.

The basic body forms one unit with the pivoting element by the basic body being connected to the pivoting element undetachably but pivotably.

The connector according to the present invention comprises at least one mount for at least one sensor, which mount may be arranged at the pivoting element. This means that a sensor connected to the pivoting element via the mount is also located in a predefined relative position with respect to the basic body due to the predefined relative position of the pivoting element with respect to the basic body. In other words, the connector according to the present invention brings about a predefined positioning of the pivoting element, of the basic body and of the sensor with respect to one another.

In particular, the basic body may be connected to a ventilation device directly or via a connection element and, because of this, it may form a ventilation system.

In particular, the pivoting element may be connected to a user on a second side directly, for example, with the use of a mouthpiece or a tube. The pivoting element may have conical connectors, for example, ones according to the ISO Standard 5356, for this purpose.

The pivoting element provided according to the present invention is arranged on its user side especially as close as possible to the user in order to minimize the dead space of a corresponding global system. Provisions may be made for a module, for example, a filter, to be arranged between the pivoting element and the user.

In particular, the mount of the pivoting element may have conical connectors, for example, ones according to the ISO Standard 5356, for connection to a sensor.

The pivoting element provided according to the present invention makes it possible to reduce pulling and/or compressive forces imparted by a user to the connector according to the present invention, which develop, for example, due to movement or repositioning of a user. The pivoting element is pivotable for this purpose about at least one axis with respect to the basic body or to at least a part of the basic body. For example, the pivoting element may be pivotable with respect to a guiding element of the basic body. The basic body and/or the pivoting element may have a swivel joint for this purpose.

In particular, the pivoting element may be pivotable horizontally and/or vertically with respect to a radial axis of an opening of the basic body. The pivoting element may be connected for this purpose to the basic body by a swivel joint, for example, a hinge or a ball-and-socket joint or a bearing. The swivel joint may connect an opening of the basic body to an opening of the pivoting element, so that the pivoting element can be rotated about the opening of the basic body or along the opening of the basic body.

The mount is configured especially as an integral part of the basic body of the connector being presented. The dead space of a global system comprising the connector is minimized by the integration of the mount into the basic body of the connector being presented, because a volume of dead space, which is an additional volume in addition to the connector and is typical in the case of an arrangement of a sensor by means of a connection module to be provided in addition to a connector, is avoided due to the fact that the mount and the basic body are combined in a three-dimensionally compact form.

The dimensions in space of the connector being presented are especially compact due to the integration of the mount into the basic body, so that a lever action of a lever between the connector and a user, which is amplified by a pulling and/or compressive force acting on the connector, is minimized.

The mount provided according to the present invention may be configured, for example, as a measuring window, into which a sensor is to be inserted or can be inserted. Further, the mount may have a cuvette, which comprises a structure with which a respective sensor can be connected or is to be connected.

Provisions are made according to the present invention for the connector to be configured as an elbow and for the pivoting element to be arranged as a bent element in relation to the basic body. As an alternative, the connector may be configured as a linear adapter and an air stream flowing through the basic body and the pivoting element may flow only along a horizontal axis.

An especially ergonomic configuration of the connector being presented, which minimizes tensile and/or pressure loads by the user, can be obtained due to a pivoting element arranged as a bent element with respect to the basic body to be provided according to the present invention, i.e., a pivoting element that is arranged, for example, at an angle between 1° and 179° and especially between 1° and 89° with respect to a radial axis of an opening of the basic body.

An especially high or strong volume flow or, compared to the air stream flowing through the basic body and the pivoting element, a low resistance, which is especially suitable for the ventilation of users with weak respiratory muscles, can be obtained by a linear configuration of the connector being presented, in which an air stream flowing through the basic body and the pivoting element flows only along a horizontal axis or a symmetry axis.

Furthermore, provisions may be made for the connector to have at least one flow element, which is configured to dam up an air stream flowing through the connector up to a predefined minimum pressure or minimum dynamic pressure. The minimum pressure may be predefined here by a defined “pressure over flow” characteristic.

An air stream flowing through the connector can be dammed up by a flow element such that a minimum pressure is reached for a measurement, for example, for a pressure difference measurement, especially a volume flow measurement, for determining a respiratory minute volume or for a pressure measurement for determining an airway pressure. The flow element may be arranged for this purpose horizontally or vertically in the connector and be configured, for example, as an arrangement of plates extending in parallel.

Provisions may, furthermore, be made for the flow element to be movable in at least some areas and to be configured to transform an air stream arriving at the flow element into an air stream flowing according to a predefined curve.

The value of a flow resistance to be provided by the flow element can be predefined or adapted by an at least partially movable flow element, for example, a flow flap, for example, with the use of a spring element. The flow element itself may consist for this purpose of an elastic material, for example, a plastic or comprise at least one spring element, for example, a mechanical spring.

In particular, a flow resistance, which changes, for example, as a function of a deflection angle of the flow element, can be provided by a movable flow element, so that, for example, an especially high flow resistance is provided in case of a small deflection angle of the flow element in order to provide a minimum dynamic pressure, which is necessary for a measurement by means of a pressure difference sensor.

Furthermore, provisions may be made for the at least one mount to comprise at least two measuring windows or pressure taps, for example, lines, which carry air flowing through respective measuring windows or openings to a sensor. In addition, two measuring windows may be provided for the carbon dioxide measurement by means of, for example, infrared radiation.

A flow to a sensor that is or can be arranged in or at the mount can be predefined by means of a mount, in which pressure taps are provided in fixed predefined positions, so that the sensor will correspondingly determine standardized and exact measured values.

The connector being provided or a sensor to be arranged at the connector is used especially for volume flow measurement to determine a respiratory minute volume, for pressure measurement to determine an airway pressure or for carrying out a measuring function to determine a carbon dioxide concentration in a breathing gas.

Provisions may, furthermore, be made for the at least one mount to be rigidly or pivotably connected to the pivoting element of the connector.

A pivoting range that is additional to the pivotable connection provided according to the present invention between the pivoting element and the basic body or an additional degree of freedom for the movement of the connector relative to a user may be provided by a pivotable mount or by an at least partially pivotably configured mount. Provisions may be made, in particular, for the basic body to be configured such that it is pivotable about a first axis, for example, a symmetry axis of an opening of the pivoting element, and for the mount or at least a part of the mount to be configured such that it is pivotable about another axis, for example, an axis extending vertically with respect to the symmetry axis.

Provisions are made according to the present invention for the at least one mount to be arranged in the at least one pivoting element. As an alternative or in addition, provisions may be made for the at least one mount to be arranged in the at least one basic body.

Further, provisions may be made for the at least one mount to arrange the at least one sensor at the connector such that the latter is suitable for carrying out a main stream measurement.

It is possible by means of the mount provided according to the present invention to predefine a position of a sensor to be arranged in or at the mount in the connector and, as a result of this, relative to additional components of a global system, for example, a ventilation device or a tube. In particular, the mount may be integrated in the connector such that a sensor to be arranged in or at the mount detects a main stream flowing through the connector and analyzes same correspondingly.

In particular, provisions may be made for the at least one mount to be arranged per se at a predefined position in a main stream of a ventilation device.

Provisions may, furthermore, be made for the at least one pivoting element to be connected to the at least one basic body such that the connector will have a minimal volume.

The connector according to the present invention is especially suitable for integrating in space a plurality of elements of a ventilation device in a predefined, preferably minimal space. It is possible due to the compact configuration in space of the connector according to the present invention to minimize a dead space, i.e., the part of a ventilation system, through which breathing gas flows during an exhalation and during an inhalation. The percentage of exhaled breathing gas is minimized by a minimal dead space in a volume of fresh breathing gas, which is fed to a user, so that the percentage of fresh breathing gas that is fed to the user is maximized.

Furthermore, provisions may be made for the at least one pivoting element to be connected to the at least one basic body by means of at least one swivel joint pivotable about at least one axis.

A swivel joint hinge, for example, a ring coupling or a ball-and-socket joint, makes possible a movement of the basic body provided according to the present invention relative to the pivoting element provided according to the present invention about at least one axis. The swivel joint may have, for example, a rigid part and a movable part for this purpose, in which case, for example, the rigid part may be connected to the pivoting element. In particular, the swivel joint is configured as an integral part of the pivoting element and/or of the basic body.

Furthermore, provisions may be made for the basic body and/or for the pivoting element and/or for the at least one mount to have at least one additional swivel joint pivotable about at least one axis.

A plurality of degrees of freedom may be provided for a movement of the connector according to the present invention relative to a user by an additional swivel joint or by a plurality of swivel joints. Provisions may be made, in particular, for each swivel joint of a plurality of swivel joints to be pivotable about a respective axis, so that the connector is to be pivoted or can be pivoted by the plurality of swivel joints about a plurality of axes.

Furthermore, provisions may be made for at least one flow element to be arranged in the connector.

Furthermore, provisions may be made for the connector to comprise at least one sensor and for the at least one sensor to be configured to determine an air mass flow and/or a carbon dioxide concentration of an air stream flowing through the connector.

In particular, the at least one sensor may be configured as a pressure difference sensor. Provisions may be made for this purpose for the mount provided according to the present invention to have at least two measuring windows or pressure taps, through which the respective one pressure column of an air stream flowing through the connector is to be fed to the pressure difference sensor. Based on the two pressure columns, it is possible to infer the flow properties of the air stream flowing through the connector by means of the pressure difference sensor. The pressure taps may be connected, for example, to a line system for supplying the pressure difference sensor with air, so that the pressure taps are also used to arrange the pressure difference sensor at the connector.

Further, the sensor provided according to the present invention may be configured to detect an air mass flow, i.e., a volume of an air stream flowing through the connector per unit of time.

Further, the sensor provided according to the present invention may be configured to detect a carbon dioxide concentration, for example, in percent (%) or in mg/L, in an air stream flowing through the connector. The sensor may comprise for this purpose, for example, an infrared element or any other element suitable for the analysis of an optical density and/or of a gas pressure.

Provisions may, furthermore, be made for a sensor to be a bidirectional sensor.

A gas flowing through the connector according to the present invention can be analyzed by means of a bidirectional sensor both during the flow in the inhalation direction and during the flow in the exhalation direction.

The connector according to the present invention is intended, in particular, for use during the mechanical ventilation of a user, for example, during the treatment of airway diseases.

The invention being presented pertains, furthermore, to a ventilation system for ventilating a user, wherein the ventilation system has a configuration of the connector according to the present invention, and the connector is connected to a ventilation device of the ventilator.

The connector according to the present invention is used especially to provide the ventilation system according to the present invention.

Further steps improving the present invention appear from the following description of some exemplary embodiments of the present invention, which are shown in the figures. All the features and/or advantages, including design details and arrangements in space, which appear from the claims, from the description or from the drawings, may be essential for the present invention both in themselves and in the different combinations. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a side view of a possible configuration of the connector;

FIG. 2 is a side view of the connector from FIG. 1 with a flow element;

FIG. 3 is a side view of the connector from FIG. 1 with a flow flap;

FIG. 4 is a side view of a possible configuration of the connector, in which the connector is configured as a linear module;

FIG. 5 is a side view of a possible configuration of the connector according to the present invention with a mount provided in a basic body of the connector; and

FIG. 6 is a view of a possible configuration of the ventilation system according to the present invention.

### DESCRIPTION OF PREFERRED EMBODIMENTS

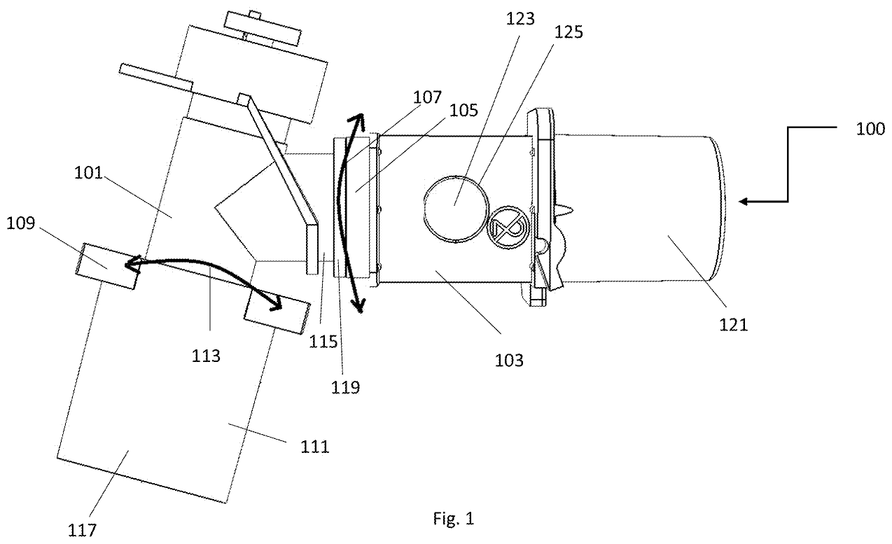

Referring to the drawings, elements having the same function and mode of operation are designated by the same reference numbers in FIGS. 1 through 6.

FIG. 1 shows a connector 100. The connector 100 comprises a pivoting element 101 and a basic body 103 defining a gas stream flow passage for flow of a gas stream the connector 100.

The connector 100 is configured as an angle connector, so that an air (gas) stream flowing through the connector 100 is deflected at an angle and the gas stream is not flowing through the connector 100 along a straight or horizontal plane.

The basic body 103 can be pivoted with respect to the pivoting element 101 by a first swivel joint 105, i.e., it is arranged rotatably along a horizontal or essentially horizontal rotation axis, as it is indicated by a first arrow 107.

An essentially horizontal rotation axis may be sloped in this case, for example, in a range between 1° and 25° with respect to the horizontal rotation axis.

Furthermore, the pivoting element 101 can be pivoted by a second swivel joint 109 with respect to a user connector 111, i.e., it is arranged rotatably along another axis of rotation, which is, for example, at an angle with respect to the horizontal axis, as it is indicated by a second arrow 113. For example, a tube, a filter or a mask of a patient, which may be configured as a part of the pivoting element 101, may be connected at the user connector 111.

Through the two rotation axes or the swivel joints 105 and 109, the connector 100 relieves a ventilation device connected to the connector 100, for example, via the connector piece 121, of a pulling or compressive force imparted by a movement of a user connected to the pivoting element 103 via the user connector 111. In other words, the connector 100 makes possible a movement of the user along the rotation axes with respect to the ventilation device connected to the connector 100, without pulling or compressive forces being transmitted thereby to the ventilation device.

The pivoting element 101 is connected directly to the basic body 103 on a first side 115.

The connector 100 may be connected to a user on a second side 117 via the user connector 111.

The basic body 103 is connected directly to the first side 115 of the pivoting element 101 on a third side 119.

The swivel joint 105 is provided here to connect the third side 119 of the basic body 103 to the first side 115 of the pivoting element 101. The swivel joint 105 may be configured completely as a part of the pivoting element 101 or it may be configured with a first part as a part of the basic body 103 and with a second part as a part of the pivoting element 101. As an alternative, the first swivel joint 105 may be configured as a part of the basic body 103.

The basic body 103 is connected on a fourth side 121 to a ventilation device.

Furthermore, the basic body 103 comprises a mount (a sensor mount) 125, by means of which a sensor can be arranged at the connector 100 in order to analyze an air stream flowing through the connector 100. The mount 125 is used to arrange an optical sensor, for example, an infrared sensor, for detecting, for example, a carbon monoxide concentration and/or a carbon dioxide concentration. The mount 125 comprises for this purpose a transparent element 123, which is permeable to rays of the optical sensor.

Due to the fact that the position of the mount 125 in the basic body 103 is predefined as a fixed position and the position of the connector 100 in a respective ventilation system is predefined as a fixed position, the sensor can only be arranged in a fixed predefined position in or relative to a respective ventilation device or user in the ventilation system. Position-related errors of measurement of the sensor, which are due, for example, to the measuring length of measured gas to be measured being longer or shorter with respect to a calibration length or an original length, are correspondingly minimized or avoided.

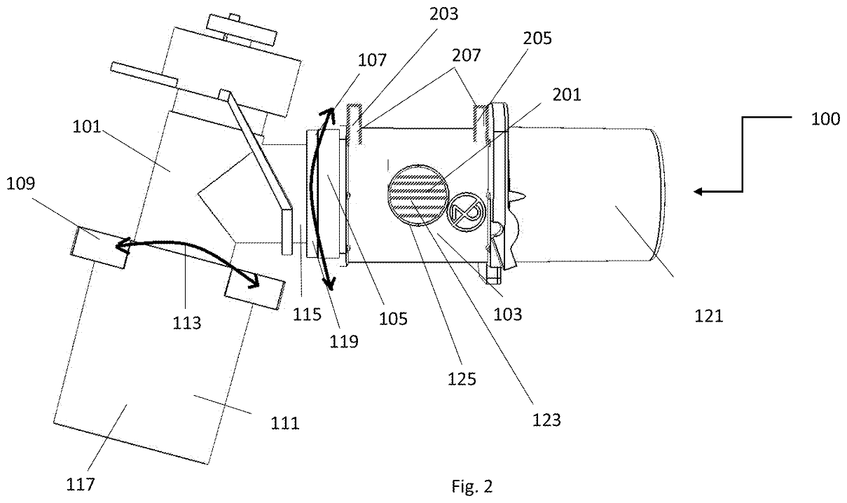

FIG. 2 shows the connector 100 with a flow element 201, through which air flowing through the connector 100 is at least partially blocked/throttled in the connector 100, so that a predefined minimum dynamic pressure becomes established during the operation of the connector 100 and a sensor arranged at the connector 100, for example, a pressure difference sensor, can be used to detect flow properties of the air stream flowing through the connector 100.

The flow element 201 shown in FIG. 2 passes through the basic body 103, especially along an overall length of the basic body 103, and is shown in FIG. 2 only in an area visible through the transparent element 123.

The flow element 201 may be configured for damming up, at least partially blocking and/or throttling dynamic pressure for an air stream, which flows from a user side or from the first side 115 through the connector 100. As an alternative or in addition, the flow element 201 may be configured for building up dynamic pressure for an air stream, which flows from the device side or from the fourth side 121 through the connector 100, so that flow properties of a user air stream and/or of a device air stream can be determined.

Further, the basic body 103 shown in FIG. 2 comprises a second mount 207 for arranging a sensor. The second mount 207 comprises a first pressure tap 203 at a first measuring window and a second pressure tap 205 arranged adjacent to the first pressure tap 203 at a second measuring window 205. The first pressure tap 203 and the second pressure tap 205 are connected to the sensor via a connection system, not shown, as a result of which the sensor is connected to the connector 100 and is arranged at this such that air flowing through the first pressure tap 203 and through the second pressure tap 205 is sent to the sensor, and the sensor, which may be, for example, a pressure difference sensor, can determine flow properties of an air stream flowing through the connector 100 on the basis of the corresponding air pressures or pressure differences between respective pressure columns.

A dynamic pressure (pressure drop), which causes air flowing through the connector 100 to flow at least partially to the sensor and causes the sensor to be able to detect properties of the air stream flowing through the connector 100, is generated by the flow element 201 in the area of the first pressure tap 203 and of the second pressure tap 205. The flow element 201 is configured such that the sensor is supplied at least with a minimal pressure necessary for a measurement and a resistance for the air stream flowing through the connector 100 remains minimal. The flow element 201 may be configured for this purpose, for example, as an arrangement of plates arranged especially parallel to one another, as it is shown in FIG. 2.

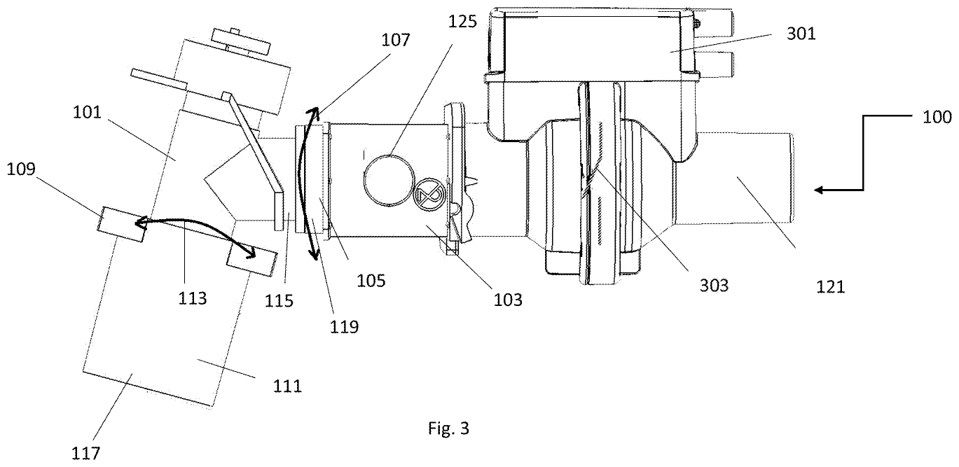

In FIG. 3, the connector 100 comprises a flow element 303 in the form of a flow flap and a mount 301 for arranging a pressure difference sensor at the connector 100 by means of a connection system, for example, a number of measuring lines.

The mount 301 may comprise, for example, a first pressure tap 203 and a second pressure tap 205, as it is shown in FIG. 2. The mount 301 may, of course, also comprise any additional, technically suitable pressure tap for providing air columns for a pressure difference measurement.

The flow element 303 is configured as a flow flap arranged partially movably in the connector 100, so that the flow element 303 moves as a function of an air stream flowing through the connector 100. This means that the flow element 303 moves away from the pivoting element 101 in case of an air stream flowing from the first side 115 through the connector 100 and to the pivoting element 101 in case of an air stream flowing from the fourth side 121 through the connector 100. The flow element 303 moves now as a function of a pressure of a respective incoming air stream, so that an air stream flowing behind the flow element 303 flows with flow characteristic flowing according to a predefined characteristic. The flow element makes it correspondingly possible to tap both an inspiratory pressure and an expiratory pressure.

In particular, the flow element 303 may be configured such that it dams up or blocks/at least partially blocks an air stream flowing with a low pressure through the connector 100 more strongly than an air stream flowing with a high pressure, so that the air stream flowing with lower pressure is dammed/blocked up to a minimum pressure and, as a result of this, the flow element 303 makes it possible to pass on the pressure to a pressure sensor.

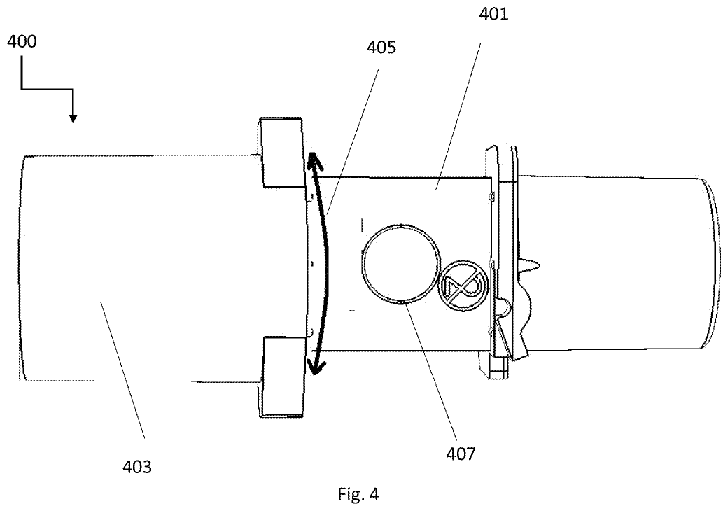

FIG. 4 shows a connector 400. The connector 400 is configured as a linear module or linear connector, so that an air stream flowing through the connector 400 is not introduced at an angle, and the air stream correspondingly flows along a straight or horizontal plane through the connector 400. A basic body 401 of the connector 400 is arranged rotatably with respect to a pivoting element 403 analogously to the mechanisms described in connection with FIGS. 1 through 3, as it is indicated by the arrow 405. A swivel joint may be arranged for this purpose at the basic body 401 or at the pivoting element 403.

A sensor may be arranged here, for example, at a mount 407, as it was already described with reference to FIGS. 1 through 3.

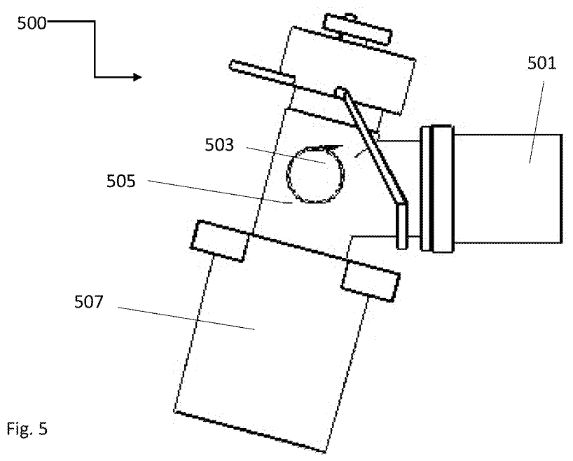

FIG. 5 shows a connector 500 according to the present invention, whose pivoting element 505 has a mount 503 for arranging a sensor at the connector 500. Due to the mount 503 being integrated into the pivoting element 505, a sensor arranged in the mount 503 can be arranged at an especially short distance to a user, as a result of which a dead space of a corresponding ventilation system is minimized. The pivoting element 505 is arranged here rotatably or pivotably with respect to a basic body 501, as it is already described analogously with reference to FIGS. 1 through 3. The pivoting element 505 is arranged for this purpose at the basic body 501 directly and pivotably, i.e., rotatably along a rotation axis via a swivel joint of the basic body 501 or via a swivel joint of the pivoting element 505.

Furthermore, the pivoting element 505 is arranged pivotably by a second swivel joint with respect to a user connector 507, i.e., along an additional rotation axis. For example, a tube, a filter or a mask of a patient may be connected at the user connector 507.

Through the two rotation axes or the swivel joints, the connector 500 relieves a sensor arranged at the mount 503 of a pulling or compressive force imparted by a movement of a user connected to the pivoting element 505 via the user connector 507, so that pulling and compressive forces acting on the sensor are minimized.

For example, a sensor may be arranged, for example, at the mount 503, as was already described with reference to the mount 125 according to FIG. 1.

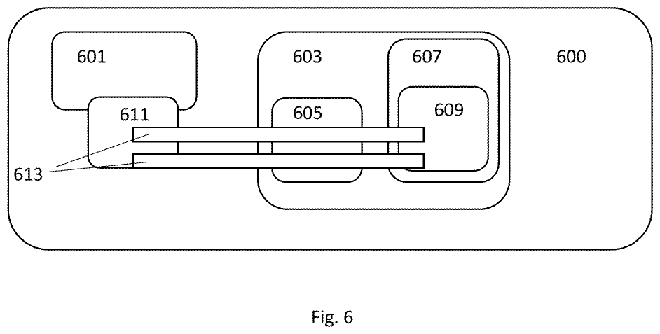

FIG. 6 shows a ventilation system 600. The ventilation system 600 comprises a ventilation device 601, a connector 603 with a basic body 605, with a pivoting element 607 and with a mount 609 provided in the pivoting element 607. A sensor 611 is arranged at the mount 609 for the analysis of breathing gas flowing through the ventilation system via a connection system 613 of the sensor 611. The connection system 613 correspondingly connects a measuring unit of the sensor 611 to the mount 609. The connection system 613 comprises in this case two lines, through which the respective one pressure column is sent from the connector 603 to the measuring unit of the sensor 611, so that the measuring unit can determine properties of an air stream flowing through the connector 603.

The pivoting element 607 is to be connected and can be connected to a user. The position of the pressure taps of the sensor 611 is predefined as a fixed position by the position of the mount 609, so that the flow path of an air stream flowing through the ventilation system 600 from the ventilation device 601 to the sensor 611 and from a user to the sensor 611 remains constant, for example, when the sensor 611 is changed or the ventilation device 601 is changed. Errors of measurement or measured value shifts of measured values determined by the sensor 611 due to position-related flow differences are correspondingly avoided with the use of the connector 603.

While specific embodiments of the invention have been shown and described in detail to illustrate the application of the principles of the invention, it will be understood that the invention may be embodied otherwise without departing from such principles.

### LIST OF REFERENCE NUMBERS

100 Connector
101 Pivoting element
103 Basic body
105 First swivel joint
107 First arrow
109 Second swivel joint
111 User connector
113 Second arrow
115 First side
117 Second side
119 Third side
121 Fourth side
123 Transparent element
125 Mount
201 Linear flow element
203 First pressure tap
205 Second pressure tap
207 Second mount
301 Mount
303 Flow element
400 Connector
401 Basic body
403 Pivoting element
405 Arrow
407 Mount
500 Connector
501 Basic body
503 Mount
505 Pivoting element
507 User connector
600 Ventilation system
601 Ventilation device
603 Connector
605 Basic body
607 Pivoting element
609 Mount
611 Sensor
613 Connection system

What is claimed is:

1. A connector for arranging at least one sensor at a ventilation system, the connector comprising:
a ventilator connection basic body for connection to at least one ventilation device;
a user connection pivoting element for connection to a user, the user connection pivoting element being connected, on a first side, directly and undetachably to the ventilator connection basic body and pivotable about at least one axis with respect to at least a part of the ventilator connection basic body to protect the ventilator connection basic body from pulling and compressive forces imparted by the user; and
a sensor mount for at least one sensor, the sensor mount being arranged at the user connection pivoting element, wherein the ventilator connection basic body is arranged in a bent form with respect to the pivoting element to provide a connector elbow configuration.

2. A connector in accordance with claim 1, further comprising a flow element for at least partially blocking gas flow of a gas stream flowing through the connector up to a predefined minimum pressure.

3. A connector in accordance with claim 2, wherein the flow element is movable in at least some areas and is configured to transform the gas stream arriving at the flow element into a gas stream flowing according to a predefined curve.

4. A connector in accordance with claim 2, wherein the flow element is arranged such that the flow element extends horizontally or vertically in the connector.

5. A connector in accordance with claim 1, wherein the sensor mount comprises at least two pressure taps for determining a pressure difference in an gas stream flowing through the connector.

6. A connector in accordance with claim 1, wherein the sensor mount is connected rigidly or pivotably to the ventilator connection basic body.

7. A connector in accordance with claim 1, wherein the sensor mount is configured to arrange the at least one sensor at the connector to carrying out a main stream measurement.

8. A connector in accordance with claim 1, wherein the user connection pivoting element is connected to the ventilator connection basic body such that the connector has a minimal flow volume.

9. A connector in accordance with claim 1, wherein the user connection pivoting element is connected directly to the ventilator connection basic body by means of at least one swivel joint pivotable about at least one axis.

10. A connector in accordance with claim 9, wherein:
the user connection pivoting element has at least one additional swivel joint pivotable about at least one axis; or
the ventilator connection basic body has at least one additional swivel joint pivotable about at least one axis; or
the sensor mount has at least one additional swivel joint pivotable about at least one axis; or
any combination of the user connection pivoting element has at least one additional swivel joint pivotable about at least one axis, and the ventilator connection basic body has at least one additional swivel joint pivotable about at least one axis, and the sensor mount has at least one additional swivel joint pivotable about at least one axis.

11. A connector in accordance with claim 1, further comprising a sensor as the at least one sensor, wherein the sensor is configured to determine an air mass flow of a gas stream flowing through the connector or a carbon dioxide concentration of a gas stream flowing through the connector or both an air mass flow of an gas stream and a carbon dioxide concentration of a gas stream flowing through the connector.

12. A connector in accordance with claim 11, wherein the sensor is a bidirectional sensor or an optical sensor or both a bidirectional sensor and an optical sensor.

13. A ventilation system for ventilating a user, the ventilation system comprising:
a ventilator with a ventilation device; and
a connector connected to the ventilation device, the connector comprising:
a ventilator connection basic body connected to the ventilation device;
a user connection pivoting element for connection to the user, the user connection pivoting element being connected, on a first side, directly and undetachably to the ventilator connection basic body and pivotable about at least one axis with respect to at least a part of the ventilator connection basic body to protect the ventilator connection basic body from pulling and compressive forces imparted by the user; and
a sensor mount for at least one sensor, the sensor mount being arranged at the user connection pivoting element, wherein the ventilator connection basic body is arranged in a bent form with respect to the pivoting element to provide a connector elbow configuration.

14. A ventilation system in accordance with claim 13, further comprising a flow element for blocking gas flow of a gas stream flowing through the connector up to a predefined minimum pressure.

15. A ventilation system in accordance with claim 14, wherein the flow element is movable in at least some areas and is configured to transform the gas stream arriving at the flow element into a gas stream flowing according to a predefined curve.

16. A ventilation system in accordance with claim 13, wherein the sensor mount comprises at least two pressure taps for determining a pressure difference in an gas stream flowing through the connector.

18. A ventilation system in accordance with claim 13, wherein the user connection pivoting element is connected directly to the ventilator connection basic body by means of at least one swivel joint pivotable about at least one axis.

19. A ventilation system in accordance with claim 18, wherein:
the user connection pivoting element has at least one additional swivel joint pivotable about at least one axis; or
the ventilator connection basic body has at least one additional swivel joint pivotable about at least one axis; or
the sensor mount has at least one additional swivel joint pivotable about at least one axis; or
any combination of the user connection pivoting element has at least one additional swivel joint pivotable about at least one axis, and the ventilator connection basic body has at least one additional swivel joint pivotable about at least one axis, and the sensor mount has at least one additional swivel joint pivotable about at least one axis.

20. A ventilation system in accordance with claim 13, further comprising a sensor as the at least one sensor, wherein the sensor is configured to determine an air mass flow of a gas stream flowing through the connector or a carbon dioxide concentration of a gas stream flowing through the connector or both an air mass flow of an gas stream and a carbon dioxide concentration of a gas stream flowing through the connector.
